(19) United States
(12) Patent Application Publication
(10) Pub. No.: US 2018/0322952 A1
(43) Pub. Date: Nov. 8, 2018

(54) DECISION SUPPORT ARBITRATION SYSTEM AND METHOD OF OPERATION THEREOF

(71) Applicant: KONINKLIJKE PHILIPS N.V., EINDHOVEN (NL)

(72) Inventors: MICHAEL L ERNANDEZ, CHESHIRE, CT (US); HERBERT PITTMAN, MERIDEN, CT (US); ERIC PAUL WIGFORSS, WALLINGFORD, CT (US)

(21) Appl. No.: 15/773,744

(22) PCT Filed: Oct. 26, 2016
(86) PCT No.: PCT/IB2016/056423

§ 371 Date: May 4, 2018

Related U.S. Application Data
(60) Provisional application No. 62/251,422, filed on Nov. 5, 2015.

(51) Int. Cl. G16H 50/20

(52) U.S. Cl. CPC G16H 50/20

(57) ABSTRACT

Disclosed is a clinical-care decision support (CCDS) system (100, 400). The CCDS system may include at least two controllers (102, 410) which may determine parameter conditions (PCs) for each of a plurality of advisors (104); determine, for each corresponding advisor, whether to perform advisor processing or exception processing based upon the determined PCs for each advisor; form advisor information (AI), for each corresponding advisor, when it is determined to perform advisor processing for the corresponding advisor; form exception information (EI), for each corresponding advisor, when it is determined to perform exception processing for the corresponding advisor; and/or may generate decision support information (DSI) based on at least one of the AI and the EI.

The present system relates to a decision support system such as a clinical care system (CCS) including an arbitration system for determining information such as decision support information (DSI) and, more particularly, to a decision support system including an arbitrator which determines DSI for decision support to assist decision makers in the use of corresponding systems and the like and a method of operation thereof.

Typically, decision support systems are relied upon to provide for example point-of-care decision support information to assist users such as clinicians and the like using the decision support systems. In a clinical environment, the decision support information may include information such as an analysis of a subject's (e.g., a patient's) condition and recommended care steps. One such decision support system is known as the VentAssist™ system which is a software system that provides point-of-care decision support to assist users such as respiratory-care clinicians with analysis of patient condition and logical future (e.g., next) acts for care. VentAssist™ includes a collection of individual advisory algorithms each of which may develop recommendations based on a given one or more input parameters. Unfortunately, these individual advisory algorithms may give conflicting advice and/or advice which may be out-of-step with each other. Accordingly, embodiments of the present system may overcome these and other disadvantages for example of conventional decision support systems.

The system(s), device(s), method(s), arrangements(s), user interface(s), computer program(s), processes, etc. (hereinafter each of which will be referred to as system, unless the context indicates otherwise), described herein address problems in prior art systems.

In accordance with embodiments of the present system, there is disclosed a clinical-care decision support system which may include: at least two controllers which may: determine parameter condition (PCs) for each of a plurality of advisors; determine, for each corresponding advisor, whether to perform advisor processing or exception processing based upon the determined PCs for each advisor; form advisor information (AI), for each corresponding advisor, when it is determined to perform advisor processing for the corresponding advisor; form exception information (EI), for each corresponding advisor, when it is determined to perform exception processing for the corresponding advisor; and/or may generate decision support information (DSI) based on at least one of the AI and the EI.

It is further envisioned that the system may include a display controlled by at least one controller which may render the generated DSI. The system wherein the at least two controllers are configured to determine parameter conditions for at least pressure support, ventilation and oxygenation advisors. It is further envisioned that at least one controller is configured to render DSI instructing to connect one or more sensors to the controller. It is also envisioned that the system may include sensors which may generate corresponding sensor information (SI), wherein at least one controller may determine the PCs based at least in part upon the SI. In accordance with embodiments of the present system, a further controller may control each of the plurality of advisors to generate the corresponding DSI based at least in part upon the parameter rule information for each of the plurality of advisors. The system may further include an arbitrator controlled by a further controller and which may generate the DSI further in accordance with parameter rule information for each of the plurality of advisors.

In accordance with embodiments of the present system, there is disclosed a clinical-care support (CCS) method performed by a clinical-care decision support system having at least two controllers, the CCS method controlled by the at least two controllers and may include acts of: determining parameter condition (PCs) for each of a plurality of advisors; determining, for each corresponding advisor, whether to perform advisor processing or exception processing based upon the determined PCs for each advisor; forming advisor information (AI), for each corresponding advisor, when it is determined to perform advisor processing for the corresponding advisor; forming exception information (EI), for each corresponding advisor, when it is determined to perform exception processing for the corresponding advisor; and/or generating decision support information (DSI) based on at least one of the AI and the EI.

In accordance with embodiments of the present system, at least one controller may perform an act of rendering the generated DSI upon a display. It is also envisioned that the method may further include an act of the at least two controllers determining parameter conditions for at least pressure support, ventilation and oxygenation advisors. The method may include an act of at least one controller rendering DSI instructions to connect one or more sensors to the controller. The method may further include an act of generating, by sensors, sensor information (SI), wherein at least one controller may determine the PCs based at least in part upon the SI. The method may further include an act of controlling, by a further controller, each of the plurality of advisors to generate the corresponding DSI based at least in part upon parameter rule information for each of the plurality of advisors. It is also envisioned that the method may further include an act of controlling an arbitrator to generate the DSI further in accordance with parameter rule information for each of the plurality of advisors.

In accordance with embodiments of the present system, there is disclosed a computer program stored on a computer readable memory medium, the computer program may be configured to control at least two controllers of a clinical-care decision support system to perform a clinical-care support (CCS) method. It is envisioned that the computer program may include a program portion that may be configured to: determine parameter condition (PCs) for each of a plurality of advisors; determine, for each corresponding advisor, whether to perform advisor processing or exception processing based upon the determined PCs for each advisor; form advisor information (AI), for each corresponding advisor, when it is determined to perform advisor processing for the corresponding advisor; form exception information (EI), for each corresponding advisor, when it is determined to perform exception processing for the corresponding advisor; and/or generate decision support information (DSI) based upon at least one of the AI and the EI.

It is further envisioned that the program portion may be configured to render the generated DSI upon a display of the system. It is also envisioned that the program portion is further configured to determine parameter conditions for at least pressure support, ventilation and oxygenation advisors. It is further envisioned that the program portion may be configured to render DSI instructing to connect one or more sensors to the controller. It is also envisioned that the program portion may be further configured to generate sensor information (SI) and may determine the PCs based at least in part upon the SI. It is also envisioned that the program portion may be configured generate the corresponding DSI based at least in part upon parameter rule information for each of the plurality of advisors.

The present invention is explained in further detail in the following exemplary embodiments and with reference to the figures, where identical or similar elements are partly indicated by the same or similar reference numerals, and the features of various exemplary embodiments being combinable. In the drawings:

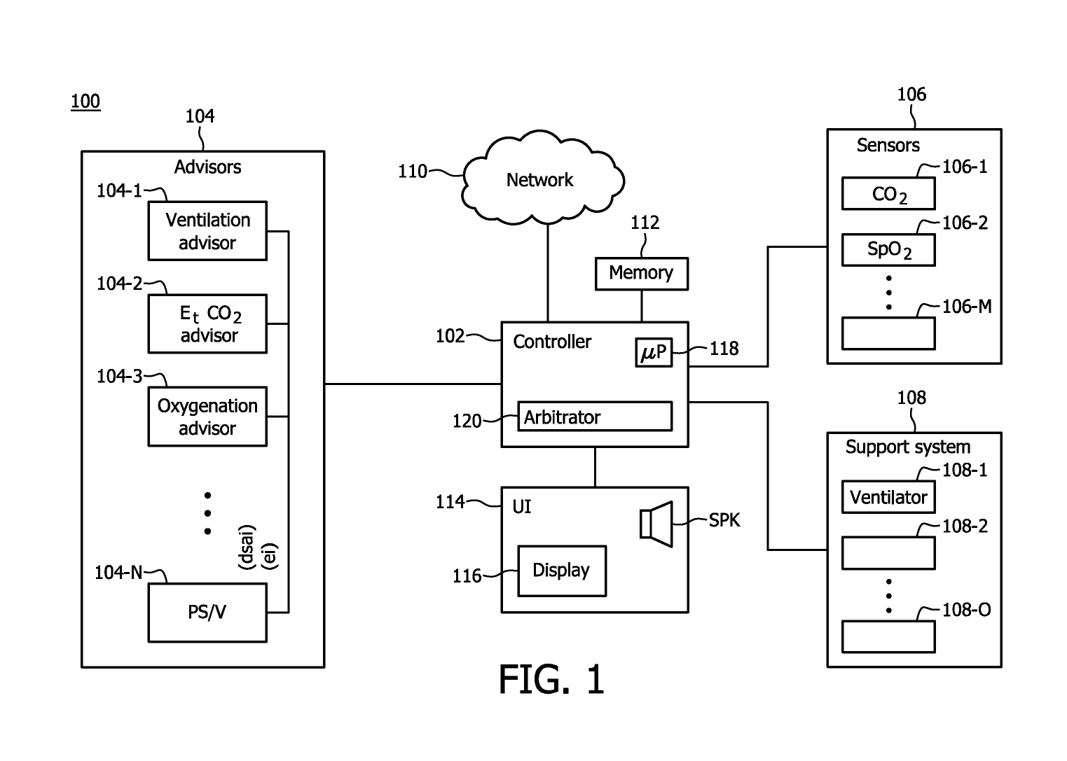

FIG. 1 shows a schematic block diagram of a portion a portion of a CCS operating in accordance with embodiments of the present system;

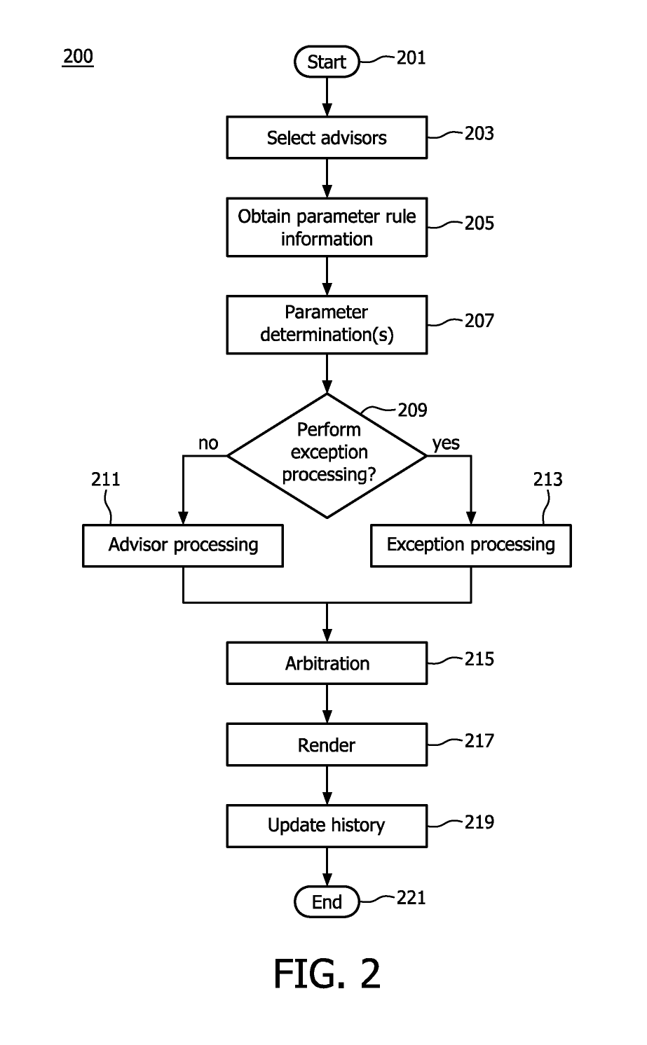

FIG. 2 shows a functional flow diagram that illustrates a decision support process performed in accordance with embodiments of the present system;

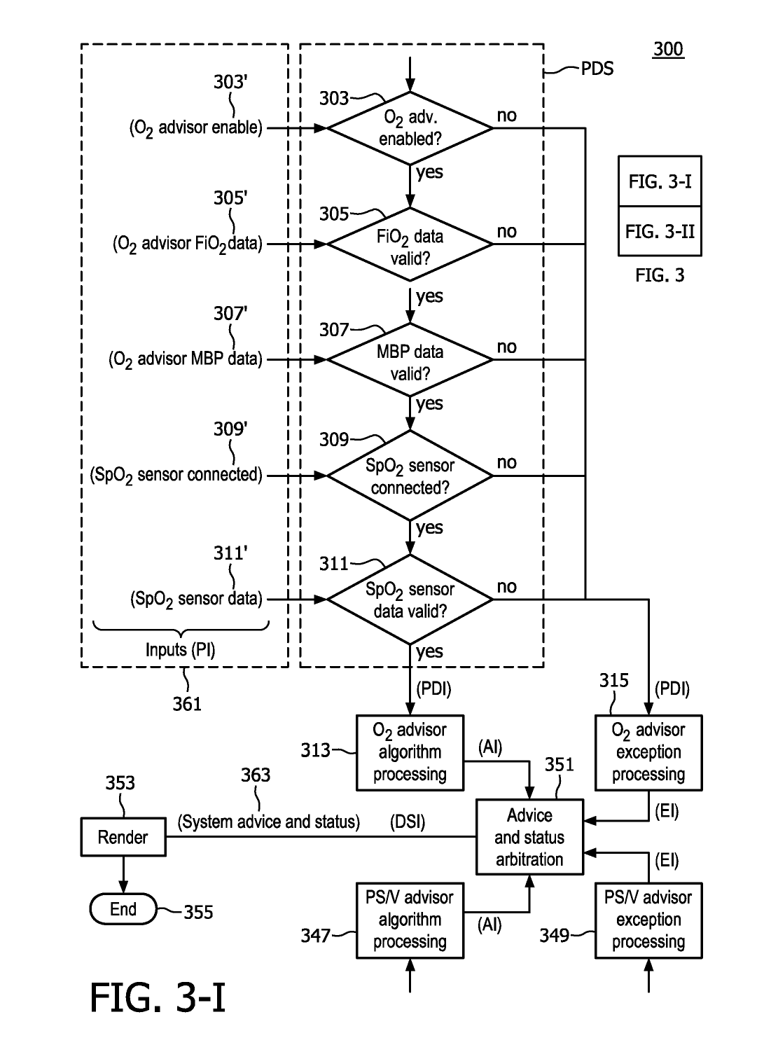

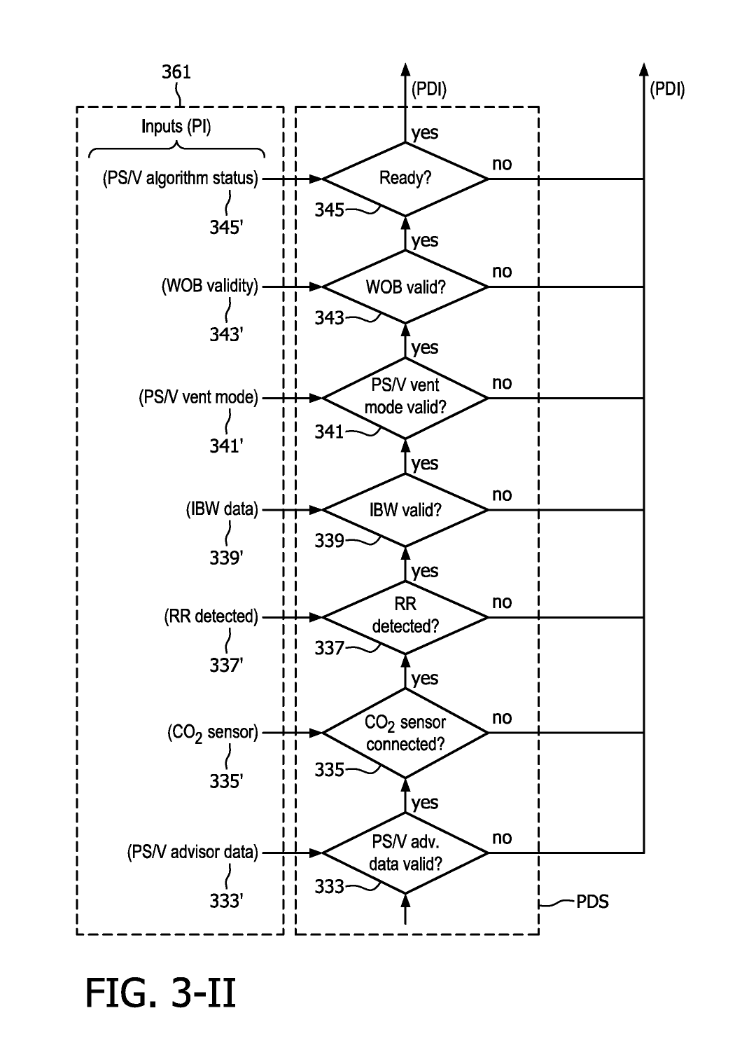

FIG. 3 shows functional flow diagram including a decision tree that illustrates a decision support process performed in accordance with embodiments of the present system; and

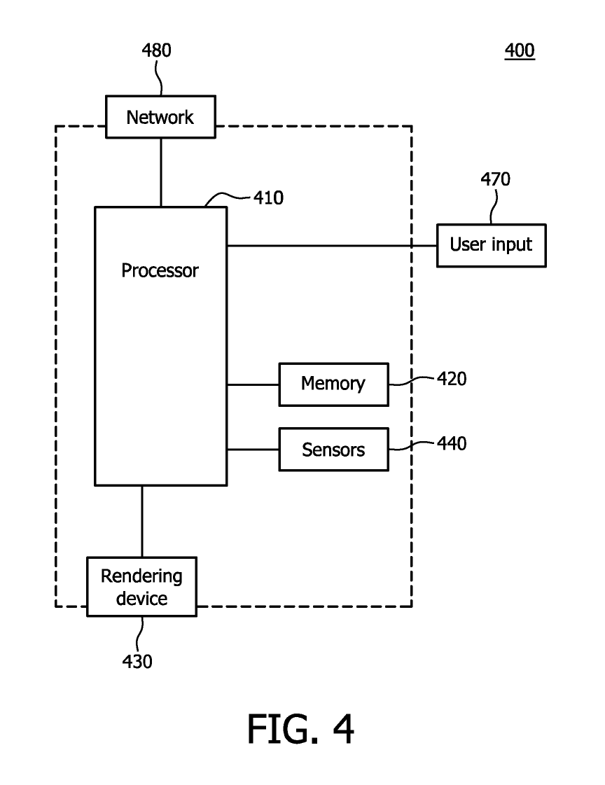

FIG. 4 shows a portion of a system in accordance with embodiments of the present system.

The following are descriptions of illustrative embodiments that when taken in conjunction with the following drawings will demonstrate the above noted features and advantages, as well as further ones. In the following description, for purposes of explanation rather than limitation, illustrative details are set forth such as architecture, interfaces, techniques, element attributes, etc. However, it will be apparent to those of ordinary skill in the art that other embodiments that depart from these details would still be understood to be within the scope of the appended claims. Moreover, for the purpose of clarity, detailed descriptions of well-known devices, circuits, tools, techniques, and methods are omitted so as not to obscure the description of the present system. It should be expressly understood that the drawings are included for illustrative purposes and do not represent the entire scope of the present system. In the accompanying drawings, like reference numbers in different drawings may designate similar elements. The term and/or and formatives thereof should be understood to mean that only one or more of the recited elements may need to be suitably present (e.g., only one recited element is present, two of the recited elements may be present, etc., up to all of the recited elements may be present) in a system in accordance with the claims recitation and in accordance with one or more embodiments of the present system.

FIG. 1 shows a schematic block diagram of a portion of a clinical care system (CCS) 100 (hereinafter system 100 for the sake of clarity) operating in accordance with embodiments of the present system. The system 100 may include one or more of a controller 102 (e.g., a processor, microcontroller, etc.), advisors 104, sensors 106, a support systems 108, a network 110, a memory 112, an arbitrator 120, and a user interface (UI) 114 one or more of which may communicate with each other using any suitable methods (e.g., wired and/or wireless methods) and/or may be implemented using any suitable method such as hardware, software and/or firmware methods. The system 100 may form critical care decisions which may be rendered, stored, etc.

The controller 102 may control the overall operation of the system 100 and may include one or more processors (e.g., at least one microprocessor such as microprocessor 118), logic devices, gates, etc. The network 110 may include any suitable network such as a wired and/or wireless network, a system buss, etc. For example, the network 110 may include a local area network (LAN), a wide area network (WAN), the Internet, a telephony network, etc. One or more of the controller 102, the advisors 104, the sensors 106, the support systems 108, the memory 112, the arbitrator 120, and the user interface (UI) 114 (e.g., such as a graphical user interface (GUI)) may communicate with each other (or portions thereof) via the network 110.

The memory 112 may include any suitable non-volatile memory which may store information used by, and/or generated by the system 100 and/or the user. For example, the memory 112 may store operating instructions, settings, parameters, etc. used by the system. Thus, for example, the memory may store arbitration instructions, clinical decision support instructions, clinical decision advice, advisor algorithms, arbitrator decision table(s) such as arbitration decision rules (ADR) and the like as described herein.

The UI 114 may include any suitable UI with which a user may interact with the system 100. For example, the UI may include one or more of a speaker (SPK), a haptic device, a display 116, etc. The display 116 may include any suitable display such as a touch-screen display which may render, for example, information generated by the system 100 and/or may receive user entries and/or selections. In accordance with embodiments of the present system, the display 116 may be local or remote from the system 100. For example, in accordance with embodiments of the present system, the display 116 may include a display of mobile computing device which may communicate with the controller 102 via the network 110. Accordingly, it is envisioned that a user such as a clinician may interact with the system via an application of the present system for example operating on a suitable device such as a mobile computing device (e.g., smart-phone, a tablet, a laptop computer, and/or the like).

The sensors 106 may include one or more sensors of the system such as sensors 106-1 through 106-M (generally 106-x) which may provide corresponding sensor information (SI). The sensors 106-x may include sensors such as carbon dioxide (CO₂) sensors 106-1, peripheral capillary oxygen saturation for an estimation of the oxygen saturation level (SpO₂) sensors 106-2, oxygen (O₂) sensors, pressure sensors such as one or more of a blood pressure sensor, an airway pressure sensor and a barometric pressure sensor, temperature sensors, flow sensors (e.g., side-stream flow, proximal flow, etc.), proximity sensors, engagement sensors (e.g., sensors which detect when a device is coupled), etc. As envisioned, that the use of an arbitrator is not dependent on the form or source of the inputs. As such, different sensors may provide input for the arbitrator depending on the embodiment. For example, the output of any one or more suitable sensors may be provided to the arbitrator and/or advisor to be incorporated into an arbitrator based clinical support system in accordance with embodiments of the present system as long as the outputs generated are compatible with other system components. Moreover although a given implementation of the arbitrator may be provided by software running on a processor, a design using in accordance with embodiments of the present system may also be implemented in hardware or a hardware and software implementation as described further herein. The sensors 106-x may further include sensors which may provide support information (SI, such as decision support information (DSI)) related to a sensed physiological condition (e.g., blood oxygenation, heart rate, blood pressure, blood CO₂, etc.) of a subject-of-interest (SOI) such as a patient, a test environment, an animal, etc., as may be desired. The sensors 106-x may provide corresponding SI to the controller 102 for further processing. Moreover, it is envisioned that the controller 102 may request SI from one or more selected sensors 106-x of the sensors 106 and/or the sensors may continuously provide SI as may be desired. The SI may form at least a part of parameter information (PI) as may be discussed herein.

The support system 108 may provide support to the SOI such as ventilation support to the SOI. For example, the support system 108 may include one or more support systems 108-1 through 108-O (generally 108-x). The support system 108-1 may include a ventilator whose settings may be controlled by the controller 102 which may be operative to change ventilator settings based upon information generated by embodiments of the present system. Further, the support system 108 may include one or more of the sensors 106-x. For example, the ventilator 108-1 may include ventilation setting sensors such as a CO_{2 (CO}2) sensor and the like which may provide corresponding SI to the controller 102. Moreover, the support system 108 may provide parameter information such as SI, settings, and/or parameter conditions (e.g., the ventilator 108-1 may provide information related to actual and/or set pressure assist levels, etc.), etc. In accordance with embodiments of the present system, it is envisioned that the present system may be suitably applied in a system for example with one more of the following characteristics including a system where there are algorithms that are utilized for processing inputs to the system and one or more outputs are generated that may be independent of one another. Further, in these systems, the outputs of these algorithms which may operate independent of one another is collectively processed and based on a set of rules to generate a combined and/or prioritized advisor output. Accordingly while described herein with regard to a medical application, present system may also be suitably applied in other systems such as in applications including in military, automotive and aerospace systems.

The advisors 104 may include at least one advisor such as advisors 104-1 through 104-N (generally 104-x). The advisors 104-x may include advisors such as a ventilation advisor 104-1, a partial pressure/maximal concentration of carbon dioxide (CO2) advisor (EtCO2) advisor 104-2, an oxygenation (O2) advisor 104-3, and a pressure ventilation and support (PS/V) advisor 104-N. Each of the advisors 104-x may include one or more decision support algorithms (DSAs) which may determine decision support advice (e.g., clinical decision support advice) and form corresponding advisor information (AI). In accordance with embodiments of the present system, each of the DSAs may obtain desired information (e.g., parameter information, SI, and/or the like as may be desired by the corresponding DSA as may be set in system settings), process this information, and determine corresponding AI. The AI from one or more of the advisors 104-x may then be provided to the arbitrator 120. For example, the AI may include information from one or more advisors 104-x such as O₂ advice information from the O2 advisor 104-3 and PSN advice information from the PS/V advisor 104-N which may be included within the AI. Each advisor 104-x may further include an exception processor which may determine exception information (EI) for each corresponding selected advisor 104-x. In accordance with embodiments of the present system, the exception processing describes a set of conditions that the arbitrator may use to override other rules based processing including “Modifying Conditions” and “Override Conditions” as described herein. In accordance with embodiments of the present system, each of the advisors 104-x may operate dependently or independently of each other. For example, each of the advisors 104-x may operate independently of each other advisor 104-x. In this way, input from disparate sensors and corresponding disparate advisors (e.g., pressure support, ventilation and oxygenation advisors) which may change at given times may be flexibly utilized to form a decision support system. The arbitrator 120 may receive the AI and/or the EI from one or more of the advisors 104-x, analyze the received AI and/or EI and/or determine relevant decision support information (DSI) based upon received AI, EI, and/or parameter determinations (PDs) in accordance with arbitration decision rules (ADRs) for example as discussed herein with reference to Table 1 below and FIGS. 2 and 3. The DSI may then be rendered by a rendering device of the system such as the display 116 and/or speaker (SPK) of the system and/or may be utilized for control of a gas delivery system, such as a support system for a patient.

For example, it is envisioned that the controller 102 may determine whether the DSI is to be rendered and/or used for setting for one of the support systems 108-x. For example, in a case wherein the controller 102 determines that DSI information may be rendered, then the controller 102 may render this information on a display 116 of the system 100. However, in a case wherein the controller 102 determines that the DSI includes a setting for one of the support systems 108-x, the controller 102 may determine the corresponding support system 108-x (e.g., ventilator 108-1) and communicate with the support system 108-x (e.g., the ventilator 108-1) to apply the settings. Accordingly, the controller 102 may analyze the DSI for setting information (e.g., a flag, an instruction, a gas delivery setting, etc.) indicative of a setting for a corresponding support system 108-x and determine whether to control the corresponding support system 108-x based upon this determination.

Operation of clinical care systems (CCS) operating in accordance with embodiments of the present system is illustratively discussed with reference to FIGS. 2 and 3. FIG. 2 shows a functional flow diagram that illustrates a (clinical) decision support process 200 (hereinafter the process 200) performed in accordance with embodiments of the present system. FIG. 3 shows functional flow diagram including a decision tree that illustrates a (clinical) decision support process 300 (hereinafter the process 300) performed in accordance with embodiments of the present system. The decision tree describes rules such as parameter determinations which define parameter validity and decision support information output that may be enforced by an arbitrator operating with one or more selected advisors (e.g., a subgroup of advisors selected from a group of advisors) in accordance with embodiments of the present system.

Referring to processes (e.g., 200 and/or 300), these processes may be performed using one or more computers communicating over a network and may obtain information from, and/or store information to one or more memories which may be local and/or remote from each other. The processes (200 and/or 300) may include one of more of the following acts. It is envisioned that the acts of the processes (200 and/or 300) may be performed using CCS system operating in accordance with embodiments of the present system. Further, one or more of these acts may be combined and/or separated into sub-acts, in a case wherein desired. Further, one or more of these acts may be skipped depending upon settings and/or order changed. Moreover, one or more acts of the processes (200 and/or 300) or portions thereof may be performed serially and/or in parallel with each other. In operation, the process 200 may start during act 201 and then proceed to act 203.

During act 203, the process may, select one or more advisors to be used during the process. These advisors may be selected (e.g., by the user and/or system) as a sub-group from all available advisors or may include all available advisors. For example, the process may select at least one advisor in accordance with advisor selection information (ASI). The ASI may set forth advisors and corresponding test types such as a default test, an O₂ test, a CO₂ test, etc. For example, assuming the system includes three advisors (e.g., A, B, and C) and there are three defined test types (e.g., tests 1 through 3). Further, assuming that the ASI sets forth that test 1 may use advisors A and C, test 2 may use advisors A and B, and test 3 may use advisors A, B, and C. When a test type is selected (e.g., by the system and/or user), the system may consult the ASI to determine advisors to use and select these advisors. For example, in a case wherein test 1 is selected, the system may select advisors A and C. Similarly, in a case wherein test 2 is selected, the system may select advisors A and B; and in a case wherein test 3 is selected, the system may select advisors A, B, and C in accordance with the ASI.

Thus, in accordance with embodiments of the present system, certain advisors may be selected by a user and/or the system depending upon user and/or system settings, respectively or the system may query which advisors are available and determine which advisors to utilize based on that determination. For example, a user may select from available advisors (e.g., using any suitable method such as selections rendered on a graphical user interface (GUI)) and/or the system to select advisors based upon stored settings. It is further envisioned that the system may select advisors according to system settings automatically. For example, assuming that there are N advisors (e.g., a group of N advisors) one or more of these N advisors (e.g., all or a sub-group) may be selected by the system in accordance with system settings, as may be set by the system and/or user.

However, for illustrative purposes, as discussed herein the process may use and/or select all available advisors. Accordingly, assuming that the present system employs two advisors (e.g., a PSN advisor and an O2 advisor), the system may select both of these advisors. It is further envisioned that the process may poll for available advisors and may select them accordingly. After completing act 203, the process may continue to act 205.

During act 205, the process may obtain parameter rule information (PRI) for each of the selected advisors. The PRI may set forth at least one determination and/or action to be performed for the each of the selected advisor(s). The PRI may be obtained from a memory of the system. Thus, the process may determine PRI for each of the selected advisors. For example, in a case wherein an advisor is selected, the process may determine PRI that may be relevant for that advisor. However, in a case wherein an advisor is not selected, its PRI may not be relevant for the process at the current time. One or more of the PRIs may set forth at least one parameter determination based upon parameter information (PI) generated and/or otherwise obtained by the process for a corresponding advisor. For example, with reference to the flow diagram of FIG. 3, the PRI for the O2 advisor may set forth a series of parameter determinations (e.g., acts 303 through 311) for the O2 advisor and the PRI for the PR/V advisor may set forth a series of parameter determinations (e.g., acts 333 through 345) for the PR/V advisor, etc. Each of these parameter determinations may rely upon corresponding input PI such as shown by 303′ through 311′ and 333′ through 345′ for the O2 and PS/V advisors respectively. The PRI may further set forth rules for the advisor algorithm processing and advisor exception processing for the selected advisor(s) as desired. After completing act 205, the process may continue to act 207.

During act 207, the process may perform one or more parameter determinations as set forth by the PRI for the selected advisor(s) and form corresponding parameter determination information (PDI) for the corresponding advisor. Accordingly, the process may obtain the PI (e.g., 303′ through 311′ and 333′ through 345′) for each of the selected advisor(s) (e.g., the O2 and PS/V advisors, respectively), perform at least one corresponding parameter determination (e.g., acts 303 through 311 and 333 through 345, respectively) and determine corresponding PDI based upon results of the corresponding one or more parameter determinations. For example, with respect to the O2 advisor, the process may determine whether an O2 advisor is enabled, whether O2 advisor FiO2 data is valid, whether O2 Advisor MBP data is valid, whether an SpO2 sensor is connected, and/or whether an SpO2 sensor data is valid (e.g., see acts 303 through 311, respectively) and form corresponding PDI. Similarly, with regard to the PS/V algorithm, the process may perform one or more parameter determinations to determine whether a PSN advisor data is valid, whether a CO2 sensor is connected, whether an RR is detected, whether IBW data is valid, whether a PSN vent mode is valid, whether work-of-breathing (WOB) data is valid, and/or whether PS/V algorithm status is ready (e.g., see acts 333-345, respectively). Illustratively, acts 303 through 311 and 333 through 345 may be discussed in further detail herein with respect to the description of FIG. 3.

The process may further perform one or more other parameter determinations such as a parameter determination of whether the O2 advisor is configured/available, whether a PS/V advisor is configured/available, whether the CO2 sensor is configured/available, and/or whether a Flow Sensor is configured/available and thereby, form corresponding PDI. In accordance with embodiments of the present system, the process may form and/or update PDI to reflect the results of the parameter determinations and may be represented using any suitable method such as a binary representation, character representation, graphically, etc. The PDI may further identify an advisor corresponding to the PDI such as O2 advisor, PS/V advisor, etc., from two more available advisors. After completing act 207, the process may continue to act 209.

During act 209, the process may determine whether to perform algorithm processing for each selected advisor(s). Accordingly, in a case wherein it is determined to perform algorithm processing, then the process may continue to act 211. However, in a case wherein it is determined to perform exception processing, then the process may continue to act 213. In accordance with embodiments of the present system, the process may determine whether to perform algorithm processing based upon the results of the parameter determinations of act 207 as may be reflected by the PDI for the corresponding advisor. More particularly, the process may determine to perform algorithm processing for example when one or more of the parameter determinations, or a number that is more than a corresponding parameter threshold (e.g., 4 for the O2 advisor and 5 for the PS/V advisor, a percent of parameters, e.g., 80%, etc.), are determined in the affirmative (e.g., yes or true (1)) or vice versa. Accordingly, the process may analyze the PDI to determine the number of affirmative or negative determinations for each advisor which were determined during the parameter determinations of act 207.

During act 211, the process may perform algorithm processing for the corresponding selected advisor(s). The algorithm processing may include a plurality of predefined acts which may be performed for each corresponding advisor. These acts may be stored in a memory of the system and may be obtained by the process, when desired. The algorithm processing may be performed by one or more decision support algorithms (DSAs) which may determine advice information (AI) for each corresponding selected algorithm (e.g., which is determined to perform the algorithm processing). For example, the AI for the O2 advisor may include O2 advice information generated by the O2 advisor while the AI for the PS/V advisor may include PSN advice generated by the PS/V advisor. Each advisor may include a clinical support advisor and/or may operate simultaneously with each other. In accordance with some embodiments, the AI may be consider advice/informational text, and/or indices that may operate as separate inputs to the arbitrator, as are the conditions which determine whether the advice/informational text is valid (e.g., exception processing). For example, the states of: O2 Configuration, PS/V Configuration, SpO2 Sensor Connected, CO2 Sensor Connected and Flow Sensor Connected Inputs in addition to the validity of the advice/informational text and/or indices may be utilized to determine which advice is output. In accordance with embodiments of the present system, all of the possible combinations of these inputs may be considered, and assigned an ID which corresponds to advice/informational text and/or indices that may be considered during the algorithm processing. After completing act 211, the process may continue to act 215.

During act 213, the process may perform exception processing for the corresponding selected advisor(s). The exception processing may include a plurality of predefined acts which may be performed for each corresponding advisor and/or group of advisors. For example, with respect to the O2 advisor, O2 advisor exception processing may be performed and corresponding exception information (EI) may be generated. Similarly, with respect to the PSN advisor, PS/V exception processing may be performed and corresponding EI generated. Acts to be performed during the exception processing for each corresponding advisor may be stored in a memory of the system and may be obtained by the process as desired (e.g., such as prior to use by a corresponding advisor). For example, in a case wherein it is determined to perform O2 exception processing, the process may obtain information related to the O2 exception processing (e.g., O2 exception information) from a memory of the system. The O2 exception information may be stored in a memory of the system as desired.

In accordance with embodiments of the present system, each individual advisor may include exception processing which is outside of the scope of the arbitrator though may include processing similar to the arbitration exception processing as described further herein. After completing act 213, the process may continue to act 215.

During act 215, the process may perform arbitration processing to determine decision support information (DSI) based upon one or more of the EI and/or AI received from each of the selected advisors (e.g., PSN and O2 advisors in the present embodiments) and/or the PDI, in accordance with arbitration decision rules (ADRs). Accordingly, the DSI may include advice and/or system settings based upon cumulative results of the algorithm and/or exception processing performed for each selected advisor and/or group (e.g., two or more) advisors. More particularly, the process may obtain arbitration decision rules (ADRs) as may be shown in Table 1 below for given advisors from a memory of the system. Thereafter, the process may determine DSI in accordance with AI, EI, and/or PDI.

For example, the arbitrator may include several types of processing conditions such as normal, modifying and override. Normal condition processing for example may describe a condition whereby there are no external conditions present to change the advice/informational text and/or indices dictated by the inputs for that ID. A modifying condition may apply when some external element must be updated before processing can complete. Further, an override condition may imply that one or more inputs are present that potentially changes the advice that would normally be presented based on other inputs.

With reference to Table, 1, this table illustrates ADRs for a given group of sensors/advisors illustratively shown as an O2 sensor/advisor, PS/V advisor/sensor, SpO2 Sensor/advisor, CO2 Sensor/advisor, and Flow Sensor/advisor. However, in a case wherein other advisors are selected or otherwise determined to be available, Table 1 may reflect ADRs for these selected/available advisors. For example, ADRs for selected/available sensors/advisors may be added to the ADR table in a suitable location and/or the table may be otherwise modified wherein other sensors/advisors are selected and/or are otherwise determined to be available.

TABLE 1
ARBITRATION DECISION RULES (ADRs)

| | PARAMETER RULE INFORMATION (PRI) | | | | | | |
|---|---|---|---|---|---|---|---|
| | O2 | PS/V | SpO2 | CO2 | Flow | DECISION SUPPORT INFORMATION (DSI) | |
| ID | Sensor | Sensor | Sensor | Sensor | Sensor | Advice Text | Informational Text |
| 1 | No | No | No | No | No | Connect sensors and enter | Connect the SpO2, CO2 and Flow sensors and |
| | | | | | | data to activate system | enter MBP, Gender and Height for System advice. |
| | | | | | | (hereinafter generally | |
| | | | | | | System). | |
| 2 | No | No | No | No | Yes | Connect sensors and enter | Connect the SpO2 and CO2 sensors and enter |
| | | | | | | data to activate System. | MBP, Gender and Height for System advice. |
| 3 | No | No | No | Yes | No | Ventilation Advice & Details | Connect the SpO2 and Flow sensors and enter |
| | | | | | | | MBP, Gender and Height to receive Oxygenation |
| | | | | | | | and Pressure Support advice. |
| 4 | No | No | No | Yes | Yes | Ventilation Advice & Details | Connect the Sp02 sensor and enter MBP, Gender |
| | | | | | | | and Height to receive Oxygenation and Pressure |
| | | | | | | | Support advice. |
| 5 | No | No | Yes | No | No | Connect sensors and enter | Connect the CO2 and Flow sensors and enter |
| | | | | | | data to activate System. | MBP, Gender and Height for System advice. |
| 6 | No | No | Yes | No | Yes | Connect sensors and enter | Connect the CO2 sensor and enter MBP, Gender |
| | | | | | | data to activate System. | and Height for System advice. |
| 7 | No | No | Yes | Yes | No | Ventilation Advice & Details | Connect the Flow sensor and enter MBP, Gender |
| | | | | | | | and Height to receive Oxygenation and Pressure |
| | | | | | | | Support advice. |
| 8 | No | No | Yes | Yes | Yes | Ventilation Advice & Details | Enter MBP, Gender and Height to receive |
| | | | | | | | Oxygenation and Pressure Support advice. |
| 9 | No | Yes | No | No | No | Connect sensors and enter | Connect the SpO2, CO2 and Flow sensors and |
| | | | | | | data to activate System. | enter MBP for System advice. |
| 10 | No | Yes | No | No | Yes | Pressure Support Advice & | Connect the SpO2 and CO2 sensors and enter |
| | | | | | | Details | MBP to receive Oxygenation and Ventilation |
| | | | | | | | advice. |
| 11 | No | Yes | No | Yes | No | Ventilation Advice & Details | Connect the SpO2 and Flow sensors and enter |
| | | | | | | | MBP to receive Oxygenation and Pressure |
| | | | | | | | Support advice. |
| 12 | No | Yes | No | Yes | Yes | Pressure Support or | Connect the SpO2 sensor and enter MBP to |
| | | | | | | Ventilation Advice & Details | receive Oxygenation advice. |
| 13 | No | Yes | Yes | No | No | Connect sensors and enter | Connect the CO2 and Flow sensors and enter |
| | | | | | | data to activate System. | MBP for System advice. |
| 14 | No | Yes | Yes | No | Yes | Pressure Support Advice & | Connect the CO2 sensor and enter MBP to |
| | | | | | | Details | receive Oxygenation and Ventilation advice. |
| 15 | No | Yes | Yes | Yes | No | Ventilation Advice & Details | Connect the Flow sensor and enter MBP to |
| | | | | | | | receive Oxygenation and Pressure Support |
| | | | | | | | advice. |
| 16 | No | Yes | Yes | Yes | Yes | Pressure Support or | Enter MBP to receive Oxygenation advice. |
| | | | | | | Ventilation Advice & Details | |
| 17 | Yes | No | No | No | No | Connect sensors and enter | Connect the SpO2, CO2 and Flow sensors and |
| | | | | | | data to activate System. | enter Gender and Height for System advice. |
| 18 | Yes | No | No | No | Yes | Connect sensors and enter | Connect the SpO2, CO2 and Flow sensors and |
| | | | | | | data to activate System. | enter Gender and Height for System advice. |
| 19 | Yes | No | No | Yes | No | Ventilation Advice & Details. | Connect the SpO2 and Flow sensors and enter |
| | | | | | | | Gender and Height for System advice. |
| 20 | Yes | No | No | Yes | Yes | Ventilation Advice & Details. | Connect the SpO2, CO2 and Flow sensors and |
| | | | | | | | enter Gender and Height to receive Oxygenation |
| | | | | | | | and Pressure Support advice. |
| 21 | Yes | No | Yes | No | No | O2 Advice & Details. | Connect the CO2 and Flow sensors and enter |
| | | | | | | | Gender and Height to receive Pressure Support |
| | | | | | | | and Ventilation advice. |
| 22 | Yes | No | Yes | No | Yes | O2 Advice & Details | Connect the CO2 sensor and enter Gender and |
| | | | | | | | Height to receive Pressure Support and |
| | | | | | | | Ventilation advice. |
| 23 | Yes | No | Yes | Yes | No | O2 or Ventilation Advice & | Connect the CO2 sensor and enter Gender and |
| | | | | | | Details | Height to receive Pressure Support and |
| | | | | | | | Ventilation advice. |
| 24 | Yes | No | Yes | Yes | Yes | O2 or Ventilation Advice & | Enter Gender and Height to receive Pressure |
| | | | | | | Details | Support advice |
| 25 | Yes | Yes | No | No | No | Connect sensors and enter | Connect the SpO2, CO2 and Flow sensors for |
| | | | | | | data to activate System. | System advice. |
| 26 | Yes | Yes | No | No | Yes | Pressure Support Advice & | Connect the SpO2 and CO2 sensors to receive |
| | | | | | | Details | Oxygenation and Ventilation advice. |
| 27 | Yes | Yes | No | Yes | No | Ventilation Advice & Details | Connect the SpO2 and Flow sensors to receive |
| | | | | | | | Oxygenation and Pressure Support advice. |
| 28 | Yes | Yes | No | Yes | Yes | Pressure Support or | Connect the SpO2 sensor to receive Oxygenation |
| | | | | | | Ventilation Advice & Details | advice. |
| 29 | Yes | Yes | Yes | No | No | O2 Advice & Details | Connect the CO2 and Flow sensors to receive |
| | | | | | | | Pressure Support and Ventilation advice. |
| 30 | Yes | Yes | Yes | No | Yes | O2 or Pressure Support | Connect the CO2 sensor to receive Ventilation |
| | | | | | | Advice & Details | advice. |
| 31 | Yes | Yes | Yes | Yes | No | O2 or Ventilation Advice & | Connect the Flow sensor to receive Pressure |
| | | | | | | Details | Support advice. |
| 32 | Yes | Yes | Yes | Yes | Yes | O2, Ventilation or Pressure | System is configured and functioning properly. |
| | | | | | | Support Advice & Details | |

| | | | |
|---|---|---|---|
| 33 | Modifying Condition | N/A | MBP value should be updated. |
| | | | (Applied to table ID'S 21, 22, 23, 24, 29, 30, 31, 32) |
| 34 | Override Condition | No Respiration Detected | A No Resp Condition precludes System advice. |
| | | | (Applied to ALL table ID's) |
| 35 | Override Condition | System Related Alarm | Clear the current alarm to resume System advice |
| | | Override | generation. |
| | | | (Applied to ALL table ID's) |
| 36 | Override Condition | Highest Priority Advisor | Set ventilator to a Pressure Support mode to |
| | | Advice & Details or “Set | calculate patient effort (WOB/min). and Pressure |
| | | Pressure Support Mode” | Support Advice. |
| | | | (Applied to table ID'S 10, 12, 14, 16, 25, 26, 28, 30, 32) |
| 37 | Override Condition | Highest Priority Advisor | Patient effort cannot be calculated. Check |
| | | Advice & Details or “Set | ventilator settings. |
| | | Pressure Support Mode” | (Applied to table ID'S 10, 12, 14, 16, 25, 26, 28, 30, 32) |
| 38 | Override Condition | Highest Priority Advisor | Please wait while System acquires data for |
| | | Advice & Details or “Please | advice. |
| | | wait while System acquires | (Applied to ID'S |
| | | data for advice” | 10, 11, 12, 14, 15, 16, 25, 26, 27, 28, 30, 31) |
| 39 | Override Condition | Highest Priority Advisor | Please connect a compatible CO2 Sensor |
| | | Advice & Details or | (Applied to table ID's |
| | | “Incompatible CO2 Sensor” | 3, 4, 7, 8, 11, 12, 15, 16, 19, 20, 23, 24, 27, 28, 31, 32) |
| 40 | Override Condition | Highest Priority Advisor | Please connect a compatible Flow Sensor |
| | | Advice & Details or | (Applied to table ID's |
| | | “Incompatible Flow Sensor” | 2, 4, 6, 8, 10, 12, 14, 16, 18, 20, 22, 24, 26, 28, 30, 32) |

With reference to Table 1, the PRI may set forth conditions (e.g., O2 sensor, PS/V sensor, SPO2 Sensor, CO2 sensor, and Flow Sensor) and corresponding DSI. For example, as shown, based on which of a given set of sensors are available (e.g., shown as “Yes” or “No”), the present system may flexibly provide advice to a user (e.g., doctor treating a patient) including instructions regarding ventilation of the patient. The DSI may include Advice Text and corresponding Informational Text. The PRI may further include modifying and/or override conditions with regard to the selected/available sensors.

For example, the process may obtain previously determined PDI and match the PDI with a corresponding PRI. Thus, in a case wherein PDI includes information such as: O2 sensor=yes; PS/V sensor=no; SPO2 Sensor=no; CO2 sensor=no; and Flow Sensor=no (e.g., PDI=10000), then the process may select the row represented by ID=17 and select the corresponding Advice Text and/or corresponding Informational Text for this row (e.g., combination of selected/available sensors and form corresponding DSI. Thus, the DSI may include a message such as “Connect sensors and enter data to activate System,” as Advice Text and “Connect the SpO2, CO2 and Flow sensors and enter Gender and Height for System advice,” as Informational Text. In the same manner, in a case wherein the PDI includes information such as: O2 sensor=yes; PS/V sensor=yes; SPO2 sensor=yes; CO2 sensor=yes; and Flow sensor=yes (e.g., PDI=11111), then the process may select the row represented by ID=1 and select the corresponding Advice Text and/or Informational Text for this row (e.g., row 1). As used herein, a “yes” may represent a true or 1 condition (e.g., the sensor is available and/or the sensor data is valid) and a “no” may represent a false or 0 condition (e.g., the sensor is not available and/or the sensor data is invalid). The PDI may be formed using any suitable format such as text and/or numerical formats or the like. After completing act 215, the process may continue to act 217.

During act 217, the process may render the DSI using any suitable rendering device of the system such as a display, a speaker (e.g., using a text-to-voice conversion), etc. Accordingly, a user such as a clinician, may be informed and may take appropriate action(s) (e.g., Pressure Support and/or Ventilation Advice such as shown in Table 1). In accordance with embodiments of the present system, control of a system such as a ventilation system is provided by a clinician that acts in response to the DSI (e.g., an open-loop system). Further, in accordance with embodiments of the present system, control of the suitable system may be enacted directly in response to the DSI without requiring the clinician's interaction (e.g., closed-loop system). After completing act 217, the process may continue to act 219.

During act 219, the process may update history information to reflect information (e.g., DSI, PI, etc.) determined by the process (e.g., the DSI, the AI, the EI, PDI, etc.) and may for example thereafter store the history information in a memory of the system for later use (e.g., analysis, record maintenance, viewing, etc.). After completing act 219, the process may continue to act 221 where it ends.

With regard to the process 300, this process illustrates acts performed for each of the selected advisors independently of each other. For example, assuming that the process 300 may employ two selected advisors: O2 and PS/V advisors, then the process may include parameter determinations to be performed for each of these advisors. These parameter determinations may be similar to the parameter determinations described during acts 207 above and, therefore, may include acts 303 through 311 for the O2 advisor and acts 333 through 345 for the PSN advisor. Accordingly, the process may start at act 303 for the O2 advisor and at act 333 for the PSN advisor. These acts may be performed serially and/or in parallel with each other. In the illustrative process shown, “IBW” indicates information on an ideal body weight, “RR” indicates information on respiration rate, “FIO2” indicates information on fraction of inspired oxygen, “MBP” indicates information on mean blood pressure and “SPO2” indicates information on peripheral capillary oxygen saturation.

Moreover, acts 303 through 311 and/or 333 through 345 may form at least part of parameter determinations (PDs) that may be defined by the user and/or system. Accordingly, it is envisioned that these acts may be defined by a user and/or system and may be replaced by other acts and/or deleted entirely from parameter determinations, as desired. Further, with respect to acts 303 through 311 and 333 through 345, input data obtained for these acts (e.g., see, 303′-311′ and 333′-345′) may be obtained from one or more sources 361 as may be desired.

For example, with reference to act 303, during this act, the process may determine whether an O2 sensor/advisor is enabled and/or whether data from the sensor is valid (e.g., within predetermined guidelines). In accordance with this illustrative embodiment, in a case wherein it is determined that the 02 sensor/advisor is enabled/valid, the process may continue to act 305. However, in a case wherein it is determined that the O2 advisor is not enabled/valid, the process may continue to act 315. To determine whether the O2 advisor is enabled/valid, the process may determine for example whether an enable signal indicative of the O2 sensor/advisor being enabled is detected. Accordingly, in a case wherein this signal is not detected, the process may determine that the O2 sensor/advisor is not enabled/valid.

During act 305, the process may determine whether an O2 advisor FiO2 data is enabled/valid. Accordingly, in a case wherein it is determined that the O2 advisor FiO2 data is valid, the process may continue to act 307. However, in a case wherein it is determined that the O2 advisor FiO2 data is not valid, the process may continue to act 315. To determine whether the O2 advisor FiO2 data is valid, the process may obtain O2 advisor FiO2 data, analyze this data and thereafter determine whether this data is valid or invalid (e.g., not valid).

During act 307, the process may determine whether an O2 advisor MPB data is valid. Accordingly, in a case wherein it is determined that the O2 advisor MPB data is valid, the process may continue to act 309. However, in a case wherein it is determined that the O2 advisor MBP data is not valid, the process may continue to act 315. To determine whether the O2 advisor MPB data is enabled/valid, the process may obtain O2 advisor MBP data, analyze this data and thereafter determine whether this data is valid, invalid, enabled and/or disabled.

For example, during act 309, the process may determine whether an SpO2 sensor is connected. Accordingly, in a case wherein it is determined that the SpO2 sensor is connected, the process may continue to act 311. However, in a case wherein it is determined that the SpO2 sensor is not connected, the process may continue to act 315. To determine whether the SpO2 sensor is connected, the process may obtain an SpO2 connection signal and determine whether this signal indicates whether the SpO2 sensor is connected or not connected.

During act 311, the process may determine whether SpO2 sensor data is valid. Accordingly, in a case wherein it is determined that the SpO2 sensor data is valid, the process may continue to act 313. However, in a case wherein it is determined that the SpO2 sensor data is not valid, the process may continue to act 315. To determine whether the SpO2 sensor data is valid, the process may obtain SpO2 sensor data, analyze this data and thereafter determine whether this data is valid or invalid.

With reference to the SP/V advisor, during act 333, the process may determine whether PSN advisor data is valid. Accordingly, in a case wherein it is determined that the PS/V advisor data is valid, the process may continue to act 335. However, in a case wherein it is determined that the PS/V advisor data is not valid, the process may continue to act 349. To determine whether the PS/V advisor data is valid, the process may obtain PS/V advisor data, analyze this data and thereafter determine whether this data is valid or invalid (e.g., not valid).

During act 335, the process may determine whether a CO2 sensor is connected. Accordingly, in a case wherein it is determined that the CO2 sensor is connected, the process may continue to act 337. However, in a case wherein it is determined that the CO2 sensor is not connected, the process may continue to act 349. To determine whether the CO2 sensor is connected, the process may obtain a CO2 connection signal and determine whether this signal indicates whether the CO2 sensor is connected (or not).

During act 337, the process may determine whether an RR is detected. Accordingly, in a case wherein it is determined that the RR is detected, the process may continue to act 339. However, in a case wherein it is determined that the RR is not detected, the process may continue to act 349. To determine whether the RR is detected, the process may obtain an RR signal. Accordingly, in a case wherein the RR signal is detected, the process may determine that the RR is detected. However, in a case wherein the RR signal is not detected, the process may determine that the RR is not detected.

During act 339, the process may determine whether IBW data is valid. Accordingly, in a case wherein it is determined that the IBW data is valid, the process may continue to act 341. However, in a case wherein it is determined that the IBW data is not valid, the process may continue to act 349. To determine whether the IBW data is valid, the process may obtain IBW data, analyze this data and thereafter determine whether this data is valid or not valid.

During act 341, the process may determine whether PS/V vent mode information is valid. Accordingly, in a case wherein it is determined that the PS/V vent mode information is valid, the process may continue to act 343. However, in a case wherein it is determined that the PS/V vent mode information is not valid, the process may continue to act 349. To determine whether the PS/V vent mode information is valid, the process may obtain a PS/V vent mode signal, analyze this signal and thereafter determine whether this signal is indicative of the PSN vent mode being valid or invalid.

During act 343, the process may determine whether WOB data is valid. Accordingly, in a case wherein it is determined that the WOB data is valid, the process may continue to act 345. However, in a case wherein it is determined that the WOB data is not valid, the process may continue to act 349. To determine whether the WOB data is valid, the process may obtain WOB data, analyze this data and thereafter determine whether this data is valid or invalid (e.g., not valid).

During act 345, the process may determine whether PSN is ready. Accordingly, in a case wherein it is determined that the PS/V is ready, the process may continue to act 347. However, in a case wherein it is determined that the PSN is not ready, the process may continue to act 349. To determine whether the PS/V is ready, the process may obtain a PS/V is ready signal, analyze this signal and thereafter determine whether this signal is indicative of the PS/V being ready or not ready.

During act 313, the process may perform O2 Advisor algorithm processing and form corresponding AI. As this act may be similar to act 211 of process 200, a further discussion of this act is not provided for brevity however, the description of act 211 may be examined for a more thorough illustrative description of this act. After completing act 313, the process may continue to act 351. Acts 315, 347, 349, the process may be similar to acts of the process 200. As these acts may be similar to acts of the process 200, the reader is directed to the description of the similar acts of the process 200 for a more thorough description of these acts. Accordingly, a further description of the acts will not be given for the sake of brevity and clarity. After completing these acts, the process may continue to act 351.

During act 351, the process may perform an advice and status operation in based upon the received AI and/or EI and in accordance with PRI and form advice and status information (e.g., System Advice and Status) 363. This information may include clinical care information such as decision support information (DSI). As this act may be similar to act 215 of process 200, the reader is directed to the description of act 215 for an illustrative description of this act. Accordingly, a further description of act 351 will not be given for the sake of clarity. After completing act 351, the process may continue to act 353.

During act 353 the process may render advice and status information (e.g., 353) generated by the process 300 using any suitable rendering device of the system (e.g., a display, a speaker, etc.). As this act may be similar to act 217, the ready may be directed to the description of act 217 for further detail. Accordingly, a further description of act 351 will not be given for the sake of clarity. After completing act 353, the process may continue to act 355 where it ends.

FIG. 4 shows a portion of a system 400 in accordance with embodiments of the present system. For example, a portion of the present system may include a processor 410 (e.g., a controller) operationally coupled to a memory 420, a rendering device such as a display 430, sensors 440, and a user input device 470. The memory 420 may be any type of device for storing application data as well as other data related to the described operation. The application data and other data are received by the processor 410 for configuring (e.g., programming) the processor 410 to perform operation acts in accordance with the present system. The processor 410 so configured becomes a special purpose machine particularly suited for performing in accordance with embodiments of the present system.

The operation acts may include configuring a CCSS by, for example, controlling the processor 410 in accordance with embodiments of the present system. Information generated by the system 400 may include content such as clinical care support information (CCSI) and the like. The content may be provided to the user (or users) using any suitable format such as text, graphics, audio, video, etc., that may be rendered on, for example, a user interface (UI) of the present system such as on the display 430, a speaker, etc., and/or may be utilized for controlling a ventilation system.

Further, the content may then be stored in a memory of the system such as the memory 420 for later use. Thus, operation acts may include requesting, determining, providing, and/or rendering of the content generated by embodiments of the present system and/or controlling a ventilation system as described herein.

The user input 470 may include a keyboard, a mouse, a trackball, or other device, such as a touch-sensitive display, which may be stand alone or be a part of a system, such as part of a personal computer, a personal digital assistant (PDA), a mobile phone (e.g., a smart phone), a monitor, a smart- or dumb-terminal or other device for communicating with the processor 410 via any operable link. The user input device 470 may be operable for interacting with the processor 410 including enabling interaction within a UI as described herein. Clearly the processor 410, the memory 420, display 430, and/or user input device 470 may all or partly be a portion of a computer system or other device such as a client and/or server.

The methods of the present system are particularly suited to be carried out by a computer software program, such program containing modules corresponding to one or more of the individual steps or acts described and/or envisioned by the present system. Such program may of course be embodied in a computer-readable medium, such as an integrated chip, a peripheral device or memory, such as the memory 420 or other memory coupled to the processor 410.

The program and/or program portions contained in the memory 420 may configure the processor 410 to implement the methods, operational acts, and functions disclosed herein. The memories may be distributed, for example between the clients and/or servers, or local, and the processor 410, where additional processors may be provided, may also be distributed or may be singular. The memories may be implemented as electrical, magnetic or optical memory, or any combination of these or other types of storage devices. Moreover, the term “memory” should be construed broadly enough to encompass any information able to be read from or written to an address in an addressable space accessible by the processor 410. With this definition, information accessible through a network is still within the memory, for instance, because the processor 410 may retrieve the information from the network for operation in accordance with the present system.

The processor 410 is operable for providing control signals and/or performing operations in response to input signals from the user input device 470 as well as in response to other devices of a network and executing instructions stored in the memory 420. The processor 410 may include one or more of a microprocessor, an application-specific or general-use integrated circuit(s), a logic device, etc. Further, the processor 410 may be a dedicated processor for performing in accordance with the present system or may be a general-purpose processor wherein only one of many functions operates for performing in accordance with the present system. The processor 410 may operate utilizing a program portion, multiple program segments, or may be a hardware device utilizing a dedicated or multi-purpose integrated circuit. Accordingly, embodiments of the present system may provide clinical decision support information (e.g., DSI) which may assist a clinician to determine a course of treatment such as a best-course of treatment. Embodiments of the present system may employ VentAssist™ clinical decision support (CDS) software operating in accordance with embodiments of the present system to determine information which may be used in respiratory care practices to assist a clinician in determining a course of patient treatment such as a best-course of patient treatment. Accordingly, embodiments of the present system may for example combine recommendations (e.g., information) from three separate advisory algorithms; such as a ventilation advisor, an EtCO2 advisor and/or an oxygenation advisor of the VentAssist™ CDS software or the like and may provide a method to couple these and/or other advisors. Accordingly, embodiments of the present system may provide portability to form new and/or other CDS platforms.

In accordance with embodiments of the present system, an arbitrator may analyze the collective advice of individual decision support algorithms that may comprise a CDS system, and from the individual advice generated by the individual support algorithms, the arbitrator may determine relevant decision support advice which may be rendered for the use of a user such as a clinician and/or may be utilized for control of a ventilation system. The advice provided by the arbitrator may include information which may direct the user to, for example, connect additional sensors, configure other parameters, and/or change ventilator settings.

It is further envisioned that the arbitrator may include a modular software architecture which may be table driven and extensible. Adding additional algorithms and/or advisors may be added by, for example, generating and/or modifying arbitration decision rule tables and/or modifying processing logic. Further, as existing ADRs rules may be easily modified to accommodate for additional advisors, the need for rewriting an entire decision support software system may not be necessary. This can save time and reduce costs of adding advisors to decision support systems.

With regard to the ADRs (e.g., see Table 1), this table may include parameter rule information (PRI) which may define conditions under which advice from a corresponding advisor may be processed and/or rendered by the system. It is further envisioned that when minimum parameter conditions (e.g., as may be defined by PDI, etc.) are met, priority-based ordering of advice may be generated and/or rendered by the system. In accordance with embodiments of the present system, override conditions may replace and/or modify a standard set of advice which may be otherwise provided by one or more corresponding advisors.

Further, embodiments of the present system may be implemented independent of operating system type and may be operative with any suitable hardware and/or software CCS incorporating one or advisors to which may generate advice such as clinical care advice. Accordingly, embodiments of the present system may provide for a module architecture which may be ported from one platform to another and may incorporate advisor algorithms as they may become available with little or no redesign of a clinical care system.

Further variations of the present system would readily occur to a person of ordinary skill in the art and are encompassed by the following claims.

Finally, the above-discussion is intended to be merely illustrative of the present system and should not be construed as limiting the appended claims to any particular embodiment or group of embodiments. Thus, while the present system has been described with reference to exemplary embodiments such as a clinical support system, it should also be appreciated that numerous other applications, modifications and alternative embodiments may be devised by those having ordinary skill in the art without departing from the broader and intended spirit and scope of the present system as for example set forth in the claims that follow. For example, the present system may be suitably applied in environments such as in applications including in military, automotive and aerospace systems. It is also envisioned that other environments wherein a plurality of input capabilities may be provided to support one or more outputs of a system may be suitable for applications of the present system. For example, other applications where varying numbers of input information is provided to support a system output may be suitable for an application of the present system. Accordingly, the specification and drawings are to be regarded in an illustrative manner and are not intended to limit the scope of the present system including the appended claims.

In interpreting the appended claims, it should be understood that:

a) the word “comprising” does not exclude the presence of other elements or acts than those listed in a given claim;

b) the word “a” or “an” preceding an element does not exclude the presence of a plurality of such elements;

c) any reference signs in the claims do not limit their scope;

d) several “means” may be represented by the same item or hardware or software implemented structure or function;

e) any of the disclosed elements may be comprised of hardware portions (e.g., including discrete and integrated electronic circuitry), software portions (e.g., computer programming), and any combination thereof;

f) hardware portions may be comprised of one or both of analog and digital portions;

g) any of the disclosed devices or portions thereof may be combined together or separated into further portions unless specifically stated otherwise;

h) no specific sequence of acts or steps is intended to be required unless specifically indicated;

i) the term “plurality of” an element includes two or more of the claimed element, and does not imply any particular range of number of elements; that is, a plurality of elements may be as few as two elements, and may include an immeasurable number of elements; and

j) the term and/or and formatives thereof should be understood to mean that only one or more of the listed elements may need to be suitably present in the system in accordance with the claims recitation and in accordance with one or more embodiments of the present system.

1. A clinical-care decision support system, comprising:
at least two controllers configured to:
determine parameter condition (PCs) for each of a plurality of advisors;
determine, for each corresponding advisor, whether to perform advisor processing or exception processing based upon the determined PCs for each advisor;
form advisor information (AI), for each corresponding advisor, when it is determined to perform advisor processing for the corresponding advisor;
form exception information (EI), for each corresponding advisor, when it is determined to perform exception processing for the corresponding advisor; and
generate decision support information (DSI) based on at least one of the AI and the EI.

2. The system of claim 1, further comprising a display wherein at least one controller is configured to render the generated DSI upon the display.

3. The system of claim 1, wherein the at least two controllers are configured to determine parameter conditions for at least pressure support, ventilation and oxygenation advisors.

4. The system of claim 3, wherein at least one controller is configured to render DSI instructing to connect one or more sensors to the controller.

5. The system of claim 1, further comprising sensors to generate corresponding sensor information (SI), wherein at least one controller determines the PCs based at least in part upon the SI.

6. The system of claim 1, wherein a further controller controls each of the plurality of advisors to generate the corresponding DSI based at least in part upon parameter rule information for each of the plurality of advisors.

7. The system of claim 1, further comprising an arbitrator controlled by a further controller and which is configured to generate the DSI further in accordance with parameter rule information for each of the plurality of advisors.

8. A clinical-care support (CCS) method performed by a clinical-care decision support system having at least two controllers, the CCS method controlled by the at least two controllers and comprising acts of:
determining parameter condition (PCs) for each of a plurality of advisors;
determining, for each corresponding advisor, whether to perform advisor processing or exception processing based upon the determined PCs for each advisor;
forming advisor information (AI), for each corresponding advisor, when it is determined to perform advisor processing for the corresponding advisor;
forming exception information (EI), for each corresponding advisor, when it is determined to perform exception processing for the corresponding advisor; and
generating decision support information (DSI) based on at least one of the AI and the EI.

9. The CCS method of claim 8, further comprising an act of rendering, by at least one controller, the generated DSI upon a display.

10. The CCS method of claim 8, further comprising an act of the at least two controllers determining parameter conditions for at least pressure support, ventilation and oxygenation advisors.

11. The CCS method of claim 10, further comprising an act of at least one controller rendering DSI instructions to connect one or more sensors to the controller.

12. The CCS method of claim 8, further comprising an act of generating, by sensors, sensor information (SI), wherein at least one controller determines the PCs based at least in part upon the SI.

13. The CCS method of claim 8, further comprising an act of controlling, by a further controller, each of the plurality of advisors to generate the corresponding DSI based at least in part upon parameter rule information for each of the plurality of advisors.

14. The CCS method of claim 8, further comprising an act of controlling an arbitrator to generate the DSI further in accordance with parameter rule information for each of the plurality of advisors.

15. A computer program stored on a computer readable memory medium, the computer program configured to control a controller of a clinical-care decision support system to perform a clinical-care support (CCS) method, the computer program comprising:
a program portion configured to:
determine parameter condition (PCs) for each of a plurality of advisors;
determine, for each corresponding advisor, whether to perform advisor processing or exception processing based upon the determined PCs for each advisor;
form advisor information (AI), for each corresponding advisor, when it is determined to perform advisor processing for the corresponding advisor;
form exception information (EI), for each corresponding advisor, when it is determined to perform exception processing for the corresponding advisor; and
generate decision support information (DSI) based upon at least one of the AI and the EI.

16. The computer program of claim 15, wherein the program portion is further configured to render the generated DSI upon a display.

17. The computer program of claim 15, wherein the program portion is further configured to determine parameter conditions for at least pressure support, ventilation and oxygenation advisors, and wherein the program portion is further configured to render DSI instructing to connect one or more sensors to the controller.

18. (canceled)

19. The computer program of claim 15, wherein the program portion is further configured to:
generate sensor information (SI); and
determine the PCs based at least in part upon the SI.

20. The computer program of claim 15, wherein the program portion is further configured generate the corresponding DSI based at least in part upon parameter rule information for each of the plurality of advisors.

21. The CCS method of claim 8, wherein the AI includes decision support advice from the determined PCs for each of the corresponding advisors; and the EI includes a set of conditions to override the AI for at least one of the corresponding advisors.
